(19) United States
(12) Patent Application Publication
(10) Pub. No.: US 2024/0180833 A1
(43) Pub. Date: Jun. 6, 2024

(54) NANOVESICLES DERIVED FROM BACTERIA OF THE GENUS PARACOCCUS AND USE THEREOF

(71) Applicant: MD HEALTHCARE INC., Seoul (KR)

(72) Inventor: Yoon-Keun KIM, Paju-Si, Gyeonggi-do (KR)

(73) Assignee: MD HEALTHCARE INC., Seoul (KR)

(21) Appl. No.: 18/553,403

(22) PCT Filed: Mar. 31, 2022
(86) PCT No.: PCT/KR2022/004646

§ 371 Date: Sep. 29, 2023

(30) Foreign Application Priority Data
Apr. 2, 2021 (KR) 10-2021-0043407
Mar. 7, 2022 (KR) 10-2022-0029014

(51) Int. Cl. A61K 9/127; A61K 35/74; A61P 29/00

(52) U.S. Cl. CPC A61K 9/127; A61K 35/74; A61P 29/00

(57) ABSTRACT

Provided are vesicles derived from bacteria of the genus Paracoccus and a use thereof in which the vesicles efficiently inhibit pathogenic biological factor-induced secretion of inflammatory mediators which induce inflammation, and the vesicles can be advantageously used for the purpose of preparing a composition for preventing, alleviating, or treating inflammatory diseases and be used therefor.

### TECHNICAL FIELD

The present invention relates to nanovesicles derived from bacteria of the genus Paracoccus and a use thereof, and more specifically, to a composition for preventing, alleviating, or treating various inflammatory diseases using nanovesicles derived from bacteria of the genus Paracoccus.

This application claims priority to and the benefit of Korean Patent Application Nos. 10-2021-0043407 and 10-2022-0029014 filed in the Korean Intellectual Property Office on Apr. 2, 2021 and Mar. 7, 2022, respectively, and all the contents disclosed in the specification and drawings of the applications are incorporated in this application.

### BACKGROUND ART

Since the beginning of the 21st century, acute infectious diseases recognized as epidemic diseases in the past have become less important, whereas chronic inflammatory diseases accompanied by immune dysfunction caused by disharmony between humans and microbiomes have changed disease patterns as main diseases.

The development of the inflammatory diseases is accompanied by abnormalities in the immune function against external causative factors. The Th17 immune response, which secretes the interleukin (hereinafter, referred to as IL)-17 cytokine, is important for the immune response to causative factors derived from bacteria, and neutrophil inflammation due to the Th17 immune response occurs upon exposure to bacterial causative factors. Inflammatory mediators, such as tumor necrosis factor-alpha (hereinafter, referred to as TNF-α) during the development of inflammation, play an important role in inflammation and cancer development. Further, it has been recently reported that IL-6, which is secreted by bacterial causative factors, plays an important role in differentiation into Th17 cells, and chronic inflammation caused by the Th17 immune response is closely related to the development of chronic inflammatory diseases as well as cancer.

It is known that the number of microorganisms coexisting in the human body has reached 100 trillion, which is 10 times more than the number of human cells, and the number of microorganism genes is more than 100 times the number of human genes. A microbiota or microbiome refers to a microbial community including bacteria, archaea, and eukarya which are present in a given habitat, and it is known that the gut microbiota or microbiome plays an important role in human physiological phenomena and has a great influence on human health and disease through interaction with human cells.

Bacteria and archaea coexisting in our body secrete nanometer-sized vesicles in order to exchange information on genes, proteins, and the like with other cells. The mucosa forms a physical defense membrane through which particles having a size of 200 nanometers (nm) or more cannot pass, so that bacteria coexisting in the mucosa cannot pass through the mucosa, but vesicles derived from bacteria have a size of 100 nanometers or less and are absorbed into our bodies after relatively freely passing through epithelial cells via the mucosa. Bacteria-derived vesicles are secreted by bacteria, but differ from bacteria in terms of constituent components, absorption rate into the body, risk of side effects, and the like, and accordingly, the use of bacteria-derived vesicles exhibits a completely different or remarkable effect than the use of live bacteria. It has recently been revealed that pathogenic bacteria-derived vesicles absorbed in our body play an important role in the pathogenesis of metabolic diseases such as diabetes and obesity.

Bacteria of the genus Paracoccus are aerobic Gram-negative bacilli belonging to the Proteobacteria phylum, and Paracoccus denitrificans bacteria are known as bacteria fixing nitrogen in nitrates. Further, Paracoccus zeaxanthinifaciens bacteria have been recently reported as bacteria that produce zeaxanthin, which has an antioxidant action. However, there is still no report on the secretion of extracellular vesicles by bacteria of the genus Paracoccus and treatment technology using the same.

Thus, in the present invention, it was confirmed that by isolating vesicles from bacteria of the genus Paracoccus for the first time and confirming the characteristics thereof, the vesicles could be used as a composition for preventing, alleviating, or treating various inflammatory diseases.

### DISCLOSURE

### Technical Problem

As a result of intensive studies to solve the problems in the related art as described above, the present inventors confirmed that vesicles derived from bacteria of the genus Paracoccus efficiently inhibit inflammatory responses caused by pathological factors, thereby completing the present invention based on this.

An object of the present invention is to provide a composition for preventing, alleviating, or treating an inflammatory disease, comprising vesicles derived from bacteria of the genus Paracoccus as an active ingredient.

However, a technical problem to be achieved by the present invention is not limited to the aforementioned problems, and the other problems that are not mentioned may be clearly understood by a person skilled in the art from the following description.

### Technical Solution

The present invention provide a composition for preventing, alleviating, or treating an inflammatory disease, comprising vesicles derived from bacteria of the genus Paracoccus as an active ingredient.

As an exemplary embodiment of the present invention, the composition may include a pharmaceutical composition, a food composition, a cosmetic composition, an ophthalmic composition, and an inhalant composition, but is not limited thereto.

As another exemplary embodiment of the present invention, the vesicles may be secreted from Paracoccus zeaxanthinifaciens, but are not limited thereto.

As yet another exemplary embodiment of the present invention, the vesicles may have an average diameter of 10 to 1000 nm, but are not limited thereto.

As yet another exemplary embodiment of the present invention, the vesicles may be naturally secreted or artificially produced from bacteria of the genus Paracoccus, but are not limited thereto.

As yet another exemplary embodiment of the present invention, the inflammatory disease may be one or more diseases selected from the group consisting of an inflammatory eye disease, an inflammatory oral disease, an inflammatory stomach disease, an inflammatory bowel disease, an inflammatory skin disease, an inflammatory respiratory disease, an inflammatory cardiovascular disease, and an inflammatory brain disease, but is not limited thereto.

As yet another exemplary embodiment of the present invention, the inflammatory eye disease may comprise one or more diseases selected from the group consisting of macular degeneration, diabetic retinopathy, and glaucoma, but is not limited thereto.

As yet another exemplary embodiment of the present invention, the inflammatory oral disease may comprise one or more diseases selected from the group consisting of gingivitis, periodontitis, and oral cancer, but is not limited thereto.

As yet another exemplary embodiment of the present invention, the inflammatory stomach disease may comprise gastritis or gastric cancer, but is not limited thereto.

As yet another exemplary embodiment of the present invention, the inflammatory bowel disease may comprise one or more diseases selected from the group consisting of colitis, food allergies, celiac disease, colon polyps, and colorectal cancer, but is not limited thereto.

As yet another exemplary embodiment of the present invention, the inflammatory skin disease may comprise atopic dermatitis or psoriasis, but is not limited thereto.

As yet another exemplary embodiment of the present invention, the inflammatory respiratory disease may comprise one or more diseases selected from the group consisting of rhinitis, sinusitis, nasal polyps, asthma, chronic obstructive pulmonary disease, and lung cancer, but is not limited thereto.

As yet another exemplary embodiment of the present invention, the inflammatory cardiovascular disease may comprise one or more diseases selected from the group consisting of arteriosclerosis, angina pectoris, coronary artery disease, and stroke, but is not limited thereto.

As yet another exemplary embodiment of the present invention, the inflammatory brain disease may comprise one or more diseases selected from the group consisting of Alzheimer's disease, Parkinson's disease, Lou Gehrig's disease, and depression, but is not limited thereto.

Further, the present invention provides a method for alleviating or treating an inflammatory disease, the method comprising administering a composition comprising vesicles derived from bacteria of the genus Paracoccus as an active ingredient to a subject in need thereof.

In addition, the present invention provides a use of a composition comprising vesicles derived from bacteria of the genus Paracoccus as an active ingredient for preventing, alleviating or treating an inflammatory disease.

Furthermore, the present invention provides a use of vesicles derived from bacteria of the genus Paracoccus for producing a drug for preventing, alleviating or treating an inflammatory disease.

### Advantageous Effects

The present inventors confirmed that intestinal bacteria were not absorbed in the body, but vesicles derived from bacteria passed through the intestinal mucosal barrier, and were absorbed by the mucosal epithelial cells, systemically distributed, and excreted from the body through the kidneys, liver, and lungs. In addition, the present inventors confirmed that when Paracoccus zeaxanthinifaciens, which is a species of bacteria of the genus Paracoccus, was cultured ex vivo and vesicles were isolated and administered to inflammatory cells, the secretion of inflammatory mediators such as IL-6 and TNF-α by pathogenic factors that induce inflammation was significantly inhibited, and it is thus expected that vesicles derived from bacteria of the genus Paracoccus according to the present invention will be advantageously used for a composition for prevention or treatment of the inflammatory diseases caused by mediators such as IL-6 or TNF-α.

### DESCRIPTION OF DRAWINGS

FIG. 1A is a set of photographs showing the distribution patterns of Gram-negative bacteria and bacteria-derived vesicles (EV) over time after the bacteria and the vesicles were orally administered to mice, and FIG. 1B is a set of views for evaluating the ex vivo distribution patterns of the bacteria and the vesicles by harvesting blood, the kidneys, the liver, and various organs at 12 hours after orally administering the bacteria and the vesicles.

FIG. 2 is a view for evaluating whether bacteria and bacteria-derived vesicles (EV) infiltrate into intestinal mucosal epithelial cells after administering the bacteria and bacteria-derived vesicles to the intestine of a mouse (Lu: gut lumen; LP: gut lamina propria).

FIG. 3 is a result of evaluating apoptosis by treating macrophages (Raw264.7 cells) with Paracoccus zeaxanthinifaciens-derived vesicles in order to evaluate the apoptotic effects of Paracoccus zeaxanthinifaciens-derived vesicles (PZX101: Paracoccus zeaxanthinifaciens-derived vesicle).

FIGS. 4A and 4B are results of comparing the secretion level of inflammatory mediators with that of E. coli EV, which is a pathogenic vesicle, by treating macrophages (Raw264.7 cells) with Paracoccus bacteria-derived vesicles in order to evaluate the pro-inflammatory effects of Paracoccus zeaxanthinifaciens-derived vesicles, FIG. 4A compares the secretion levels of IL-6, and FIG. 4B compares the secretion levels of TNF-α (PZX101: Paracoccus zeaxanthinifaciens-derived vesicle).

FIGS. 5A and 5B are results of evaluating effects of E. coli EV on the secretion of inflammatory mediators by pretreatment with Paracoccus zeaxanthinifaciens-derived vesicles prior to treatment with E. coli EV, which is a pathogenic vesicle, in order to evaluate the anti-inflammatory effects of Paracoccus zeaxanthinifaciens-derived vesicles, FIG. 5A compares the secretion levels of IL-6, and FIG. 5B compares the secretion levels of TNF-α (PZX101: Paracoccus zeaxanthinifaciens-derived vesicle).

### BEST MODE

The present invention relates to vesicles derived from bacteria of the genus Paracoccus and a use thereof.

The present inventors confirmed that the inflammatory response caused by pathogenic factors was efficiently inhibited by treating inflammatory cells with Paracoccus zeaxanthinifaciens-derived vesicles before administering the pathogenic factors, which induce inflammation, thereby completing the present invention based on this.

As used herein, the term “extracellular vesicle,” “nanovesicle,” or “vesicle” refers to a nano-sized structure made of lipid membranes secreted by various bacteria, and in the present invention, extracellular vesicles, nanovesicles or vesicles collectively refer to all structures made of membranes naturally secreted or artificially produced by bacteria of the genus Paracoccus. The vesicles may be isolated from a culture solution including bacteria of the genus Paracoccus using one or more methods selected from the group consisting of centrifugation, ultra-high speed centrifugation, extrusion, sonication, cell lysis, homogenization, freezing-thawing, electroporation, mechanical decomposition, chemical treatment, filtration by a filter, gel filtration chromatography, free-flow electrophoresis, and capillary electrophoresis. In addition, for isolation, washing for removing impurities, and concentration of the obtained vesicles may be further performed.

In the present invention, the vesicles derived from bacteria of the genus Paracoccus may have an average diameter of 10 nm to 1000 nm, 10 nm to 900 nm, 10 nm to 800 nm, 10 nm to 700 nm, 10 nm to 600 nm, 10 nm to 500 nm, 10 nm to 400 nm, 10 nm to 300 nm, or 10 nm to 200 nm, but the average diameter is not limited thereto.

As an aspect of the present invention, the present invention provide a pharmaceutical composition for preventing or treating an inflammatory disease, comprising vesicles derived from bacteria of the genus Paracoccus as an active ingredient.

As used herein, the term “inflammatory disease” refers to a disease which is caused by exposure to a causative factor that induces inflammation, resulting in an inflammatory response, and thus induces cell damage and cell death, and includes cancer, or a degenerative brain disease, and the like which occur as a result of inflammation. The inflammatory disease may comprise, for example, an inflammatory eye disease including one or more selected from the group consisting of macular degeneration, diabetic retinopathy, glaucoma, and the like; an inflammatory oral disease comprising one or more selected from the group consisting of gingivitis, periodontitis, oral cancer, and the like; an inflammatory stomach disease comprising one or more selected from the group consisting of gastritis, gastric cancer, and the like; an inflammatory bowel disease comprising one or more selected from the group consisting of colitis, food allergies, celiac disease, colon polyps, colorectal cancer, and the like; an inflammatory skin disease comprising one or more selected from the group consisting of atopic dermatitis, psoriasis, and the like; an inflammatory respiratory disease comprising one or more diseases selected from the group consisting of rhinitis, sinusitis, nasal polyps, asthma, chronic obstructive pulmonary disease, lung cancer, and the like; an inflammatory cardiovascular disease comprising one or more selected from the group consisting of arteriosclerosis, angina pectoris, coronary artery disease, stroke, and the like; or an inflammatory brain disease comprising one or more diseases selected from the group consisting of Alzheimer's disease, Parkinson's disease, Lou Gehrig's disease, depression, and the like, but is not limited thereto.

The pharmaceutical composition according to the present invention may further include a suitable carrier, excipient, and diluent which are commonly used in the preparation of pharmaceutical compositions. The excipient may be, for example, one or more selected from the group consisting of a diluent, a binder, a disintegrant, a lubricant, an adsorbent, a humectant, a film-coating material, and a controlled release additive.

The pharmaceutical composition according to the present invention may be used by being formulated, according to commonly used methods, into a form such as powders, granules, sustained-release-type granules, enteric granules, liquids, eye drops, elixirs, emulsions, suspensions, spirits, troches, aromatic water, lemonades, tablets, sustained-release-type tablets, enteric tablets, sublingual tablets, hard capsules, soft capsules, sustained-release-type capsules, enteric capsules, pills, tinctures, soft extracts, dry extracts, fluid extracts, injections, capsules, perfusates, or a preparation for external use, such as plasters, lotions, pastes, sprays, inhalants, patches, sterile injectable solutions, or aerosols. The preparation for external use may have a formulation such as creams, gels, patches, sprays, ointments, plasters, lotions, liniments, pastes, or cataplasmas.

As the carrier, the excipient, and the diluent that may be included in the pharmaceutical composition according to the present invention, lactose, dextrose, sucrose, oligosaccharides, sorbitol, mannitol, xylitol, erythritol, maltitol, starch, acacia rubber, alginate, gelatin, calcium phosphate, calcium silicate, cellulose, methyl cellulose, microcrystalline cellulose, polyvinylpyrrolidone, water, methyl hydroxy benzoate, propyl hydroxybenzoate, talc, magnesium stearate, and mineral oil may be used.

For formulation, commonly used diluents or excipients such as fillers, thickeners, binders, wetting agents, disintegrants, and surfactants are used.

As additives of tablets, powders, granules, capsules, pills, and troches according to the present invention, excipients such as corn starch, potato starch, wheat starch, lactose, white sugar, glucose, fructose, D-mannitol, precipitated calcium carbonate, synthetic aluminum silicate, dibasic calcium phosphate, calcium sulfate, sodium chloride, sodium hydrogen carbonate, purified lanolin, microcrystalline cellulose, dextrin, sodium alginate, methyl cellulose, sodium carboxymethylcellulose, kaolin, urea, colloidal silica gel, hydroxypropyl starch, hydroxypropyl methylcellulose (HPMC), HPMC 1928, HPMC 2208, HPMC 2906, HPMC 2910, propylene glycol, casein, calcium lactate, and Primojel®; and binders such as gelatin, Arabic gum, ethanol, agar powder, cellulose acetate phthalate, carboxymethylcellulose, calcium carboxymethylcellulose, glucose, purified water, sodium caseinate, glycerin, stearic acid, sodium carboxymethylcellulose, sodium methylcellulose, methylcellulose, microcrystalline cellulose, dextrin, hydroxycellulose, hydroxypropyl starch, hydroxymethylcellulose, purified shellac, starch, hydroxypropyl cellulose, hydroxypropyl methylcellulose, polyvinyl alcohol, and polyvinylpyrrolidone may be used, and disintegrants such as hydroxypropyl methylcellulose, corn starch, agar powder, methylcellulose, bentonite, hydroxypropyl starch, sodium carboxymethylcellulose, sodium alginate, calcium carboxymethylcellulose, calcium citrate, sodium lauryl sulfate, silicic anhydride, 1-hydroxypropylcellulose, dextran, ion-exchange resin, polyvinyl acetate, formaldehyde-treated casein and gelatin, alginic acid, amylose, guar gum, sodium bicarbonate, polyvinylpyrrolidone, calcium phosphate, gelled starch, Arabic gum, amylopectin, pectin, sodium polyphosphate, ethyl cellulose, white sugar, magnesium aluminum silicate, a di-sorbitol solution, and light anhydrous silicic acid; and lubricants such as calcium stearate, magnesium stearate, stearic acid, hydrogenated vegetable oil, talc, lycopodium powder, kaolin, Vaseline, sodium stearate, cacao butter, sodium salicylate, magnesium salicylate, polyethylene glycol (PEG) 4000, PEG 6000, liquid paraffin, hydrogenated soybean oil (Lubri wax), aluminum stearate, zinc stearate, sodium lauryl sulfate, magnesium oxide, Macrogol, synthetic aluminum silicate, silicic anhydride, higher fatty acids, higher alcohols, silicone oil, paraffin oil, polyethylene glycol fatty acid ether, starch, sodium chloride, sodium acetate, sodium oleate, dl-leucine, and light anhydrous silicic acid may be used.

As additives of liquids according to the present invention, water, dilute hydrochloric acid, dilute sulfuric acid, sodium citrate, monostearic acid sucrose, polyoxyethylene sorbitol fatty acid esters (twin esters), polyoxyethylene monoalkyl ethers, lanolin ethers, lanolin esters, acetic acid, hydrochloric acid, ammonia water, ammonium carbonate, potassium hydroxide, sodium hydroxide, prolamine, polyvinylpyrrolidone, ethylcellulose, and sodium carboxymethylcellulose may be used.

In syrups according to the present invention, a white sugar solution, other sugars or sweeteners, and the like may be used, and as necessary, a fragrance, a colorant, a preservative, a stabilizer, a suspending agent, an emulsifier, a viscous agent, or the like may be used.

In emulsions according to the present invention, purified water may be used, and as necessary, an emulsifier, a preservative, a stabilizer, a fragrance, or the like may be used.

In suspensions according to the present invention, suspending agents such as acacia, tragacanth, methylcellulose, carboxymethylcellulose, sodium carboxymethylcellulose, microcrystalline cellulose, sodium alginate, hydroxypropyl methylcellulose (HPMC), HPMC 1828, HPMC 2906, HPMC 2910, and the like may be used, and as necessary, a surfactant, a preservative, a stabilizer, a colorant, and a fragrance may be used.

Injections according to the present invention may include: solvents such as distilled water for injection, a 0.9% sodium chloride solution, Ringer's solution, a dextrose solution, a dextrose+sodium chloride solution, PEG, lactated Ringer's solution, ethanol, propylene glycol, non-volatile oil-sesame oil, cottonseed oil, peanut oil, soybean oil, corn oil, ethyl oleate, isopropyl myristate, and benzene benzoate; cosolvents such as sodium benzoate, sodium salicylate, sodium acetate, urea, urethane, monoethylacetamide, butazolidine, propylene glycol, the Tween series, amide nicotinate, hexamine, and dimethylacetamide; buffers such as weak acids and salts thereof (acetic acid and sodium acetate), weak bases and salts thereof (ammonia and ammonium acetate), organic compounds, proteins, albumin, peptone, and gums; isotonic agents such as sodium chloride; stabilizers such as sodium bisulfite (NaHSO₃) carbon dioxide gas, sodium metabisulfite (Na₂S₂O₅), sodium sulfite (Na₂SO₃), nitrogen gas (N₂), and ethylenediamine tetraacetic acid; sulfating agents such as 0.1% sodium bisulfide, sodium formaldehyde sulfoxylate, thiourea, disodium ethylenediaminetetraacetate, and acetone sodium bisulfite; a pain relief agent such as benzyl alcohol, chlorobutanol, procaine hydrochloride, glucose, and calcium gluconate; and suspending agents such as sodium CMC, sodium alginate, Tween 80, and aluminum monostearate.

In suppositories according to the present invention, bases such as cacao butter, lanolin, Witepsol, polyethylene glycol, glycerogelatin, methylcellulose, carboxymethylcellulose, a mixture of stearic acid and oleic acid, Subanal, cottonseed oil, peanut oil, palm oil, cacao butter+cholesterol, lecithin, lanette wax, glycerol monostearate, Tween or span, imhausen, monolan(propylene glycol monostearate), glycerin, Adeps solidus, buytyrum Tego-G, cebes Pharma 16, hexalide base 95, cotomar, Hydrokote SP, S-70-XXA, S-70-XX75(S-70-XX95), Hydrokote 25, Hydrokote 711, idropostal, massa estrarium (A, AS, B, C, D, E, I, T), masa-MF, masupol, masupol-15, neosuppostal-N, paramount-B, supposiro OSI, OSIX, A, B, C, D, H, L, suppository base IV types AB, B, A, BC, BBG, E, BGF, C, D, 299, suppostal N, Es, Wecoby W, R, S, M, Fs, and tegester triglyceride matter (TG-95, MA, 57) may be used.

Solid preparations for oral administration include tablets, pills, powders, granules, capsules, and the like, and such solid preparations are formulated by mixing the composition with at least one excipient, e.g., starch, calcium carbonate, sucrose, lactose, gelatin, and the like. In addition to simple excipients, lubricants such as magnesium stearate and talc are also used.

Examples of liquid preparations for oral administration include suspensions, liquids for internal use, emulsions, syrups, and the like, and these liquid preparations may include, in addition to simple commonly used diluents, such as water and liquid paraffin, various types of excipients, for example, a wetting agent, a sweetener, a fragrance, a preservative, and the like. Preparations for parenteral administration include an aqueous sterile solution, a non-aqueous solvent, a suspension, an emulsion, a freeze-dried preparation, and a suppository. Non-limiting examples of the non-aqueous solvent and the suspension include propylene glycol, polyethylene glycol, a vegetable oil such as olive oil, and an injectable ester such as ethyl oleate.

The pharmaceutical composition according to the present invention is administered in a pharmaceutically effective amount. In the present invention, “the pharmaceutically effective amount” refers to an amount sufficient to treat diseases at a reasonable benefit/risk ratio applicable to medical treatment, and an effective dosage level may be determined according to factors including types of diseases of patients, the severity of disease, the activity of drugs, sensitivity to drugs, administration time, administration route, excretion rate, treatment period, and simultaneously used drugs, and factors well known in other medical fields.

The composition according to the present invention may be administered as an individual therapeutic agent or in combination with other therapeutic agents, may be administered sequentially or simultaneously with therapeutic agents in the related art, and may be administered in a single dose or multiple doses. It is important to administer the composition in a minimum amount that can obtain the maximum effect without any side effects, in consideration of all the aforementioned factors, and this may be easily determined by those of ordinary skill in the art.

The pharmaceutical composition of the present invention may be administered to a subject via various routes. All administration methods can be predicted, and the pharmaceutical composition may be administered via, for example, oral administration, subcutaneous injection, intraperitoneal injection, intravenous injection, intramuscular injection, intrathecal (space around the spinal cord) injection, sublingual administration, administration via the buccal mucosa, intrarectal insertion, intravaginal insertion, ocular administration, intra-aural administration, intranasal administration, inhalation, spraying via the mouth or nose, transdermal administration, percutaneous administration, or the like.

The pharmaceutical composition of the present invention is determined depending on the type of a drug, which is an active ingredient, along with various related factors such as a disease to be treated, administration route, the age, gender, and body weight of a patient, and the severity of diseases.

As another aspect of the present invention, the present invention provide a food composition for preventing or alleviating an inflammatory disease, comprising vesicles derived from bacteria of the genus Paracoccus as an active ingredient.

In the present invention, the food composition may be a health functional food composition, but is not limited thereto.

The vesicles derived from bacteria of the genus Paracoccus according to the present invention may be used by adding the vesicles derived from bacteria of the genus Paracoccus as is to food or may be used together with other foods or food ingredients, but may be appropriately used according to a typical method. The mixed amount of the active ingredient may be suitably determined depending on the purpose of use thereof (for prevention or alleviation). In general, when a food or beverage is prepared, the vesicles derived from bacteria of the genus Paracoccus of the present invention is added in an amount of 15 wt % or less, preferably 10 wt % or less based on the raw materials. However, for long-term intake for the purpose of health and hygiene or for the purpose of health control, the amount may be less than the above-mentioned range, and the vesicles have no problem in terms of stability, so the active ingredient may be used in an amount more than the above-mentioned range.

The type of food is not particularly limited. Examples of food to which the material may be added include meats, sausage, bread, chocolate, candies, snacks, confectioneries, pizza, instant noodles, other noodles, gums, dairy products including ice creams, various soups, beverages, tea, drinks, alcoholic beverages, vitamin complexes, and the like, and include all health functional foods in a typical sense.

The health beverage composition according to the present invention may contain various flavors or natural carbohydrates, and the like as additional ingredients as in a typical beverage. The above-described natural carbohydrates may be monosaccharides such as glucose and fructose, disaccharides such as maltose and sucrose, polysaccharides such as dextrin and cyclodextrin, and sugar alcohols such as xylitol, sorbitol, and erythritol. As a sweetener, it is possible to use a natural sweetener such as thaumatin and stevia extract, a synthetic sweetener such as saccharin and aspartame, and the like. The proportion of the natural carbohydrates is generally about 0.01 to 0.20 g, or about 0.04 to 0.10 g per 100 ml of the composition of the present invention.

In addition to the aforementioned ingredients, the composition of the present invention may contain various nutrients, vitamins, electrolytes, flavors, colorants, pectic acids and salts thereof, alginic acid and salts thereof, organic acids, protective colloid thickeners, pH adjusters, stabilizers, preservatives, glycerin, alcohols, carbonating agents used in carbonated drinks, and the like. In addition, the composition of the present invention may contain flesh for preparing natural fruit juice, fruit juice drinks, and vegetable drinks. These ingredients may be used either alone or in combinations thereof. The proportion of these additives is not significantly important, but is generally selected within a range of 0.01 to 0.20 part by weight per 100 parts by weight of the composition of the present invention.

As still another aspect of the present invention, the present invention provide an inhalant composition for preventing or treating an inflammatory disease, comprising vesicles derived from bacteria of the genus Paracoccus as an active ingredient.

In the inhalant composition of the present invention, the active ingredient may be added to the inhalant as it is, or may be used together with another ingredient, and the active ingredient may be appropriately used according to a conventional method. A mixing amount of the active ingredient may be suitably determined depending on its purpose of use (for prevention or treatment).

As an inhalant for parenteral administration, an aerosol, a powder for inhalation, or a liquid for inhalation is included, and such a liquid for inhalation may be dissolved or suspended in water or another suitable medium at the time of use. Such an inhalant is prepared in accordance with a known method. For example, a liquid for inhalation is formulated by suitably selecting a preservative (benzalkonium chloride, paraben, etc.), a coloring agent, a buffer (sodium phosphate, sodium acetate, etc.), an isotonic agent (sodium chloride, concentrated glycerin, etc.), a thickening agent (carboxyvinyl polymer, etc.), and an absorption enhancer as needed.

A powder for inhalation is formulated by suitably selecting a lubricant (stearic acid and a salt thereof, etc.), a binder (starch, dextrin, etc.), an excipient (lactose, cellulose, etc.), a coloring agent, a preservative (benzalkonium chloride, paraben, etc.), and an absorption promoter as needed.

The inhalant composition may be administered using an inhalant apparatus, the inhalant apparatus is an apparatus that can deliver the composition to a subject such as lung tissue thereof, for example, an inhaler, a nebulizer or a ventilator. To administer a liquid for inhalation, a common spray (atomizer or nebulizer) is used, and to administer a powder for inhalation, usually, an inhalation dispenser for powder medicine is used.

As yet another aspect of the present invention, the present invention provide a cosmetic composition for preventing or alleviating an inflammatory disease, comprising vesicles derived from bacteria of the genus Paracoccus as an active ingredient.

A formulation for the cosmetic composition according to the present invention may include a skin lotion, a skin softener, a skin toner, an astringent, a lotion, a milk lotion, a moisturizing lotion, a nourishing lotion, a massage cream, a nourishing cream, a mist, a moisturizing cream, a hand cream, a hand lotion, a foundation, an essence, a nourishing essence, a pack, soap, a cleansing foam, a cleansing lotion, a cleansing cream, a cleansing oil, a cleansing balm, a body lotion or a body cleanser.

A cosmetic composition of the present invention may further include a composition selected from the group consisting of water-soluble vitamins, oil-soluble vitamins, polymer peptides, polymeric polysaccharides, and sphingolipids.

The water-soluble vitamin may be any substance that is blendable with cosmetics, but examples thereof include vitamin B1, vitamin B2, vitamin B6, pyridoxine, pyridoxine hydrochloride, vitamin B12, pantothenic acid, nicotinic acid, nicotinic acid amide, folic acid, vitamin C, vitamin H, and the like, and salts thereof (thiamine hydrochloride, sodium ascorbate, and the like) or derivatives thereof (sodium ascorbic acid-2-phosphate, magnesium ascorbic acid-2-phosphate, and the like) are also included in water-soluble vitamins that may be used in the present invention. These water-soluble vitamins may be obtained by a conventional method such as microbial transformation, purification from a microbial culture, an enzyme method, or a chemical synthesis method.

The oil-soluble vitamins may be any substance that is blendable with cosmetics, but examples thereof include vitamin A, carotene, vitamin D2, vitamin D3, vitamin E (d1-α-tocopherol, d-α-tocopherol), or the like, and derivatives thereof (e.g., ascorbyl palmitate, ascorbyl stearate, ascorbyl dipalmitate, d1-α-tocopherol acetate, d1-α-tocopherol nicotinate, vitamin E, DL-pantothenyl alcohol, D-pantothenyl alcohol, pantothenyl ethylether) may also be included in the oil-soluble vitamins used in the present invention. These oil-soluble vitamins may be obtained by a conventional method such as microbial transformation, purification from a microbial culture, or enzymatic or chemical synthesis.

The polymer peptides may be any substance that is blendable with cosmetics, but examples thereof may include collagen, hydrolyzed collagen, gelatin, elastin, hydrolyzed elastin, and keratin. The polymer peptides may be purified and obtained by any conventional method such as purification from a microbial culture, an enzyme method, or a chemical synthesis method, or may generally be used by being purified from natural substances such as the dermis of a pig, a cow, or the like and silk fiber of silkworms.

The polymeric polysaccharides may be any substance that is blendable with cosmetics, and examples thereof may include hydroxyethyl cellulose, xanthan gum, sodium hyaluronate, and chondroitin sulfate or salts thereof (sodium salts). For example, chondroitin sulfate or salts thereof may generally be purified from mammals or fish and used.

The sphingolipids may be any substance that is blendable with cosmetics, and examples thereof may include ceramide, phytosphingosine, and sphingoglycolipid. The sphingolipids may be purified, by a conventional method, from mammals, fish, shellfish, yeast, or plants, or may be obtained by a chemical synthesis method.

The cosmetic composition of the present invention may include, as necessary, other ingredients mixed in conventional cosmetics along with the above essential ingredients.

Examples of additional ingredients to be mixed may include lipid components, a humectant, an emollient, a surfactant, organic and inorganic pigments, organic powder, a UV absorbent, a preservative, a sanitizer, an antioxidant, a plant extract, a pH adjuster, alcohol, pigments, flavors, a blood circulation promoter, a cooling agent, an anti-diaphoretic, and purified water.

The lipid components may include, for example, ester lipids, hydrocarbon lipids, silicone lipids, fluorine lipids, animal fats, vegetable oil, or the like.

The ester lipids may include, for example, glyceryl tri 2-ethylhexanoate, cetyl 2-ethylhexanoate, isopropyl myristate, butyl myristate, isopropyl palmitate, ethyl stearate, octyl palmitate, isocetyl isostearate, butyl stearate, ethyl linolate, isopropyl linolate, ethyl oleate, isocetyl myristate, isostearyl myristate, isostearyl palmitate, octyldodecyl myristate, isocetyl isostearate, diethyl sebacate, diisopropyl adipate, isoalkyl neopentanate, tri(capryl, capric acid)glyceryl, trimethylolpropane tri 2-ethylhexanoate, trimethylolpropane triisostearate, pentaerythritol tetra 2-ethylhexanoate, cetyl caprylate, decyl laurate, hexyl laurate, decyl myristate, myristyl myristate, cetyl myristate, stearyl stearate, decyl oleate, cetyl ricinoleate, isostearyl laurate, isotridecyl myristate, isocetyl palmitate, octyl stearate, isocetyl stearate, isodecyl oleate, octyldodecyl oleate, octyldodecyl linolate, isopropyl isostearate, cetostearyl 2-ethylhexanoate, stearyl 2-ethylhexanoate, hexyl isostearate, ethyleneglycol dioctanoate, ethyleneglycol dioleate, propyleneglycol dicaprinate, propyleneglycol di(caprylate, caprinate), propyleneglycol dicaprylate, neopentylglycol dicaprinate, neopentylglycol dioctanoate, glyceryl tricaprylate, glyceryl triundecylate, glyceryl triisopalmitate, glyceryl triisostearate, octyldodecyl neopentanoate, isostearyl octanoate, octyl isononanoate, hexyldecyl neodecanoate, octyldodecyl neodecanoate, isocetyl isostearate, isostearyl isostearate, octyldecyl isostearate, polyglycerin ester oleate, polyglycerin ester isostearate, triisocetyl citrate, triisoalkyl citrate, triisooctyl citrate, lauryl lactate, myristyl lactate, cetyl lactate, octyldecyl lactate, triethyl citrate, acetyltriethyl citrate, acetyltributyl citrate, trioctyl citrate, diisostearyl malate, 2-ethylhexyl hydroxystearate, di 2-ethylhexyl succinate, diisobutyl adipate, diisopropyl sebacate, dioctyl sebacate, cholesteryl stearate, cholesteryl isostearate, cholesteryl hydroxystearate, cholesteryl oleate, dihydrocholesteryl oleate, phytosteryl isostearate, phytosteryl oleate, isocetyl 12-stearoyl hydroxystearate, stearoyl 12-stearoyl hydroxystearate, isostearyl 12-stearoyl hydroxystearate, and the like.

The hydrocarbon lipids may include, for example, squalene, liquid paraffin, alpha-olefin oligomers, isoparaffin, ceresine, paraffin, liquid isoparaffin, polybutene, microcrystalline wax, Vaseline, and the like.

The silicone lipids may include, for example, polymethyl silicon, methylphenyl silicon, methyl cyclopolysiloxane, octamethyl polysiloxane, decamethyl polysiloxane, dodecamethyl cyclosiloxane, dimethylsiloxane/methylcetyloxysiloxane copolymers, dimethylsiloxane/methylstearoxysiloxane copolymers, alkyl-modified silicon oil, amino-modified silicon oil, and the like.

The fluorine lipids may include perfluoropolyether and the like.

The animal or vegetable oil may include avocado oil, almond oil, olive oil, sesame oil, rice bran oil, safflower oil, soybean oil, corn oil, rape flower oil, apricot kernel oil, palm kernel oil, palm oil, castor oil, sunflower oil, grape seed oil, cotton seed oil, coconut oil, tallow nut oil, wheat germ oil, rice germ oil, Shea butter, evening primrose oil, macadamia nut oil, meadow foam seed oil, yolk oil, beef tallow, hemp seed oil, mink oil, orange roughy oil, jojoba oil, candelilla wax, carnauba wax, liquid lanolin, dehydrated castor oil, and the like.

The humectant may include water-soluble low molecular humectants, oil-soluble molecular humectants, water-soluble polymers, oil-soluble polymers, and the like.

The water-soluble low molecular humectants may include serine, glutamine, sorbitol, mannitol, pyrrolidone-sodium carboxylate, glycerin, propylene glycol, 1,3-butylene glycol, ethylene glycol, polyethylene glycol B (degree of polymerization: n=2 or higher), polypropylene glycol (degree of polymerization: n=2 or higher), polyglycerin B (degree of polymerization: n=2 or higher), lactic acid, lactates, and the like.

The oil-soluble low molecular humectants may include cholesterol, cholesterol ester, and the like.

The water-soluble polymers may include carboxyvinyl polymers, polyasparaginic acid salts, tragacanth, xanthan gum, methyl cellulose, hydroxymethyl cellulose, hydroxylethyl cellulose, hydroxypropyl cellulose, carboxymethyl cellulose, water-soluble chitin, chitosan, dextrin, and the like.

The oil-soluble polymers may include, for example, polyvinyl pyrrolidone/eicosen copolymers, polyvinyl pyrrolidone/hexadecene copolymers, nitrocellulose, dextrin fatty acid ester, silicone polymers, and the like.

The emollients may include, for example, long chain cholesterylester acyl glutamate, cholesteryl hydroxystearate, 12-hydroxystearic acid, stearic acid, rosin acid, lanolin fatty acid cholesteryl ester, and the like.

The surfactants may include, for example, non-ionic surfactants, anionic surfactants, cationic surfactants, amphoteric surfactants, and the like.

The non-ionic surfactants may include self-emulsion type glycerin monostearate, propyleneglycol fatty acid ester, glycerin fatty acid ester, polyglycerin fatty acid ester, sorbitan fatty acid ester, polyoxyethylene (POE) sorbitan fatty acid ester, POE sorbit fatty acid ester, POE glycerin fatty acid ester, POE alkylethers, POE fatty acid ester, POE dehydrated castor oil, POE castor oil, polyoxyethylene/polyoxypropylene (POE/POP) copolymers, POE/POP alkylethers, polyether-modified silicone, alkanolamide laurate, alkylamine oxide, hydrated soy phospholipids, and the like.

The anionic surfactants may include fatty acid soap, α-acylsulfonate, alkyl sulfonates, alkylallyl sulfonates, alkylnaphthalene sulfonates, alkyl sulfates, POE alkylether sulfates, alkylamide sulfates, alkyl phosphates, POE alkyl phosphates, alkylamide phosphates, alkyloyl alkyltaurin salts, N-acylamino acid salts, POE alkylether carboxylates, alkyl sulfosuccinates, sodium alkyl sulfoacetates, acylated hydrolyzed collagen peptide salts, perfluoroalkyl ester phosphates, and the like.

The cationic surfactants may include, for example, alkyltrimethylammonium chloride, stearyltrimethylammonium chloride, steraryltrimethylammonium bromide, cetostearyl trimethylammonium chloride, distearyl dimethylammonium chloride, stearylaryl dimethylbenzylammonium chloride, behenyltrimethylammonium bromide, benzalkonium chloride, diethylaminoethylamide stearate, dimethylaminopropylamide stearate, quaternary ammonium salts of lanolin derivatives, and the like.

The amphoteric surfactants may include carboxybetaine, amidebetaine, sulfobetaine, hydroxysul fobetaine, amidesulfobetaine, phosphobetaine, aminocarboxylate, imidazoline derivatives, amideamine-based amphoteric surfactants, and the like.

The organic and inorganic pigments may include: inorganic pigments such as silicic acid, anhydrous silicic acid, magnesium silicate, talc, sericite, mica, kaolin, bengala, clay, bentonite, titanium dioxide-coated mica, bismuth oxychloride, zirconium oxide, magnesium oxide, zinc oxide, titanium oxide, aluminum oxide, calcium sulfate, barium sulfate, magnesium sulfate, calcium carbonate, magnesium carbonate, iron oxide, ultramarine, chromium oxide, chromium hydroxide, calamine, and combinations thereof; organic pigments such as polyamide, polyester, polypropylene, polystyrene, polyurethane, vinyl resin, urea resin, phenol resin, fluorine resin, silicon resin, acryl resin, melamine resin, epoxy resin, polycarbonate resin, divinyl benzene/styrene copolymers, silk powder, cellulose, CI pigment yellow, and CI pigment orange; and composite pigments of inorganic and organic pigments.

The organic powder may include: metallic soap such as calcium stearate; metal salts of alkyl phosphoric acid such as zinc sodium cetylate, zinc laurylate, and calcium laurylate; polymetallic salts of acylamino acid such as calcium N-lauroyl-beta-alanine, N-lauroyl-beta-alanine, and calcium N-lauroylglycine; polymetallic salts of amide sulfonates such as calcium N-lauroyl-taurine and calcium N-palmitoyl-taurine; N-acyl alkaline amino acids such as N-epsilon-lauroyl-L-lysine, N-epsilon-palmitoyl lysine, N-α-palmitoylol nitin, N-α-lauroyl arginine, and N-α-dehydrated tallow fatty acid acyl arginine; N-acyl polypeptides such as N-lauroyl glycylglycine; α-amino fatty acids such as α-aminocaprylic acid and α-aminolauric acid; polyethylene; polypropylene; nylon; polymethylmethacrylate; polystyrene; divinylbenzene/styrene copolymers; ethylene tetrafluoride; and the like.

The UV absorbents may include para-aminobenzoic acid, ethyl para-aminobenzoate, amyl para-aminobenzoate, octyl para-aminobenzoate, ethyleneglycol salicylate, phenyl salicylate, octyl salcylate, benzyl salicylate, butylphenyl salicylate, homomentyl salicylate, benzyl cinnamate, para-methoxycinnamic acid-2-ethoxylethyl, octyl paramethoxycinnamate, mono-2-ethylhexaneglyceryl diparamethoxycinnamate, isopropyl paramethoxycinnamate, diisopropyl/diisopropyl cinnamic acid ester mixtures, urocanic acid, ethyl urocanate, hydroxymethoxybenzophenone, hydroxymethoxybenzophenone sulfonic acid and salts thereof, dihydroxymethoxy benzophenone, sodium dihydroxymethoxy benzophenone disulfonate, dihydroxy benzophenone, tetrahydroxy benzophenone, 4-tert-butyl-4′-methoxydibenzoylmethane, 2,4,6-trianilino-p-(carbo-2′-ethylhexyl-1′-oxy)-1,3,5-triazine, 2-(2-hydroxy-5-methylphenyl)benzotriazole, and the like.

The sanitizers may include hinokitiol, trichloric acid, trichlorohydroxydiphenylether, chlorohexidine gluconate, phenoxyethanol, resorcine, isopropylmethylphenol, azulene, salicylic acid, zinc pyrithione, benzalkonium chloride, light sensitive element No. 301, sodium mononitroguaiacol, undecylenic acid, and the like.

The antioxidants may include butylhydroxyanisole, propyl gallate, elisorbic acid, and the like.

The pH adjusters may include citric acid, sodium citrate, malic acid, sodium malate, fumaric acid, sodium fumarate, succinic acid, sodium succinate, sodium hydroxide, sodium monohydrophosphate, and the like.

The alcohols may include higher alcohols such as cetyl alcohol.

In addition, additional ingredients to be mixed are not limited to the above examples, and any one of the above ingredients may be mixed within a range that does not adversely affect the objectives and effects of the present invention, but may range from 0.01 wt % to 5 wt % or 0.01 wt % to 3 wt % with respect to the total weight of the composition.

For lotion, paste, cream, or gel preparations of the present invention, as a carrier ingredient, animal fiber, vegetable fiber, wax, paraffin, starch, tragacanth, a cellulose derivative, polyethylene glycol, silicon, bentonite, silica, talc, zinc oxide, or the like may be used.

For powder or spray preparations of the present invention, lactose, talc, silica, aluminum hydroxide, calcium silicate, or polyamide powder may be used as a carrier ingredient. In particular, in the case of spray preparations, the composition may further include a propellant such as chlorofluorohydrocarbon, propane/butane, or dimethyl ether.

For solution or emulsion preparations of the present invention, a solvent, a solubilizing agent, or an emulsifying agent may be used as a carrier ingredient, and the carrier ingredient may be, for example, water, ethanol, isopropanol, ethyl carbonate, ethyl acetate, benzyl alcohol, benzyl benzoate, propylene glycol, 1,3-butyl glycol oil, a glycerol aliphatic ester, polyethylene glycol, or a sorbitan fatty acid ester.

For suspension preparations of the present invention, as a carrier ingredient, a liquid diluent such as water, ethanol, or propylene glycol, a suspending agent such as ethoxylated isostearyl alcohol, polyoxyethylene sorbitol ester, or polyoxyethylene sorbitan ester, micro-crystalline cellulose, aluminum methahydroxide, bentonite, agar, tragacanth, or the like may be used.

For surfactant-containing cleansing preparations of the present invention, as a carrier ingredient, an aliphatic alcohol sulfate, an aliphatic alcohol ether sulfate, a sulfosuccinate monoester, isethionate, imidazolinium derivatives, methyltaurate, sarcosinate, fatty acid amide ether sulfate, alkylamidobetaine, an aliphatic alcohol, a fatty acid glyceride, a fatty acid diethanol amide, vegetable oil, a lanolin derivative, an ethoxylated glycerol fatty acid ester, or the like may be used.

As yet another aspect of the present invention, the present invention provide an ophthalmic composition for preventing or treating an inflammatory disease, comprising vesicles derived from bacteria of the genus Paracoccus as an active ingredient.

The ophthalmic composition according to the present invention may include additives such as pharmaceutically acceptable carriers, excipients or diluents, and properties to be considered for the excipients include compatibility with cyclosporine and trehalose, bioaffinity, processing temperature, and the like, and are not limited thereto.

The ophthalmic composition according to the present invention may be prepared into a parenteral formulation such as an ophthalmic solution, an ophthalmic ointment, an injection, and an eyewash, or an oral formulation such as a tablet, a capsule and a granule, a preferred dosage form is an ophthalmic solution, and the most preferred dosage form is an eye drop.

When the ophthalmic composition is prepared as an ophthalmic solution, the ophthalmic composition may be provided in any dosage form used as an ophthalmic solution, for example, an aqueous ophthalmic solution such as an aqueous ophthalmic solution, an aqueous emulsion ophthalmic solution, a viscous ophthalmic solution and a dissolved ophthalmic solution; or a non-aqueous ophthalmic solution such as a non-aqueous ophthalmic solution and a non-aqueous emulsion ophthalmic solution.

When the ophthalmic composition is prepared as an aqueous emulsion ophthalmic solution, various additives known in the art may be included, as long as they do not impair the purpose of the invention, and for example, an isotonic agent, a buffer, a stabilizer, a pH adjusting agent, a thickener, a preservative, a chelating agent, a solubilizer, a solvent, and the like may be included. The buffer may be selected from the group consisting of a phosphate buffer, a borate buffer, a citrate buffer, a tartrate buffer, an acetate buffer (for example, sodium acetate), tromethamine and an amino acid, but is not limited thereto. Preferably, a phosphate buffer may be used. The isotonic agent may be selected from the group consisting of sugar alcohols such as sorbitol, erythritol and mannitol, sugars such as glucose, polyhydric alcohols such as glycerin, polyethylene glycol and polypropylene glycol, and salts such as sodium chloride, but is not limited thereto. The preservative may be selected from the group consisting of benzalkonium chloride, benzethonium chloride, alkyl paraoxybenzoates such as methyl paraoxybenzoate and ethyl paraoxybenzoate, benzyl alcohol, phenethyl alcohol, sorbic acid and salts thereof, thimerosal, polyquaternium, benzododecinium bromide, an oxychloro complex and chlorobutanol, but is not limited thereto. The stabilizer may be selected from the group consisting of cyclodextrin and derivatives thereof, a water-soluble polymer such as poly(vinylpyrrolidone), and a surfactant such as polysorbate 80 (Tween 80®), polysorbate 20 and tyloxapol, but is not limited thereto. The pH adjusting agent may be selected from the group consisting of hydrochloric acid, acetic acid, phosphoric acid, sulfuric acid, sodium hydroxide, potassium hydroxide, monoethanolamine, ammonia water and ammonium hydroxide, but is not limited thereto. The thickener may be selected from the group consisting of hydroxyethyl cellulose, hydroxypropyl cellulose, methyl cellulose, hydroxypropylmethyl cellulose and carboxymethyl cellulose, polyvinyl alcohol, a carbomer, povidone, a poloxamer, polycarbophil and salts thereof, but is not limited thereto. The chelating agent may be selected from the group consisting of sodium edetate, sodium citrate and condensed sodium phosphate, but is not limited thereto. The solubilizer or solvent may be selected from glycerin, DMSO, DMA, N-methylpyrrolidone, ethanol, benzyl alcohol, isopropyl alcohol, polyethylene glycol or propylene glycol with various molecular weights, and the like, but is not limited thereto. There may be some overlaps between components that can be used as the solvent or the solubilizer, and since any component may be used as either the solvent or the solubilizer, when any component acts as a solvent in the preparation, it may be regarded as a solvent, and when any component does not act as a solvent, it may be regarded as a solubilizer. Alternatively, the solubilizer may be a surfactant in some variations. Surfactant combinations including various types of surfactants may be commercially available. For example, nonionic, anionic (i.e., soap, sulfonates), cationic (i.e., CTAB), zwitterionic, polymeric, amphoteric surfactants may be used. For example, available surfactants include, but are not limited to, those having an HLB of 10, 11, 12, 13, or 14 or more. Examples of the surfactant include a polyoxyethylene product of hydrogenated vegetable oil, polyethoxylated castor oil or polyethoxylated hydrogenated castor oil, polyoxyl castor oil or derivatives thereof, polyoxyethylene-sorbitan-fatty acid ester, polyoxyethylene castor oil derivatives, and the like, but are not limited thereto. According to exemplary embodiments, the ophthalmic composition of the present invention may include 0.01 to 0.1 wt % of cyclosporine, 0.5 to 7.5 wt % of trehalose, 1 to 10 wt % of a solubilizer, 0.01 to 2 wt % of a solvent, the remainder as a buffer and an isotonic agent, based on the total weight of the composition.

The aqueous emulsion ophthalmic solution may preferably be prepared as a nanoemulsion type, and in this case, various additives known in the art may be included as long as they do not impair the purpose of the present invention, and for example, an oil, a surfactant, and the like may be included. The oil may be one or more selected from the group consisting of propylene glycol monocaprylate, propylene glycol laurate, medium chain (C8-C10) triglycerides, glyceryl-1,3-dioleate, glyceryl monooleate, and glyceryl linoleate. The surfactant may be one or more selected from the group consisting of oleoyl macrogolglycerides, linoleoyl macrogolglycerides, caprylocaproyl polyoxylglycerides, polyoxyl 35 castor oil, polyoxyl 35 hydrogenated castor oil, polyoxyl 40 hydrogenated castor oil, polyoxyl 40 hydrogenated castor oil, a condensation product of ethylene oxide with 12-hydroxystearic acid, and polysorbate 80 (Croda, UK).

The ophthalmic composition according to the present invention may be provided by being filled in a sterile container and may be provided with instructions for the use thereof, and the instructions may be physically attached to the container filled with the ophthalmic composition or to a second container in which the container is packaged, or may be packaged together within the second container.

Furthermore, the ophthalmic composition according to the present invention includes an eye drop composition (eye composition), an artificial tear composition, and the like.

The artificial tear composition of the present invention may additionally include an electrolyte, a nonionic surfactant, an antimicrobial agent, a borate and polyol complex or a low molecular weight amino acid.

The electrolyte included in the artificial tear composition is used to stimulate natural tears and includes ions included in natural tears as ingredients. For example, an electrolyte for use in the present invention includes potassium, calcium, magnesium and zinc.

As yet another aspect of the present invention, the present invention provides a method for alleviating or treating an inflammatory disease, the method comprising administering a composition comprising vesicles derived from bacteria of the genus Paracoccus as an active ingredient to a subject in need thereof.

As yet another aspect of the present invention, the present invention provides a use of a composition comprising vesicles derived from bacteria of the genus Paracoccus as an active ingredient for preventing, alleviating or treating an inflammatory disease.

As yet another aspect of the present invention, the present invention provides a use of vesicles derived from bacteria of the genus Paracoccus for producing a drug for preventing, alleviating or treating an inflammatory disease.

The term “prevention” as used herein means all actions that inhibit or delay the onset of a target disease. The term “treatment” as used herein means all actions that alleviate or beneficially change a target disease and abnormal metabolic symptoms caused thereby via administration of the pharmaceutical composition according to the present invention. The term “alleviation” as used herein means all actions that reduce the degree of parameters related to a target disease, e.g., symptoms via administration of the composition according to the present invention.

In an exemplary embodiment of the present invention, it was confirmed that, by orally administering bacteria and vesicles derived from bacteria to mice and evaluating the in vivo absorption, distribution, and excretion patterns of the bacteria and the vesicles, the bacteria were not absorbed through the intestinal mucosa, whereas the vesicles were absorbed within 5 minutes after administration, systemically distributed, and excreted through the kidneys, the liver, and the like (see Example 1).

In another exemplary embodiment of the present invention, as a result of evaluating whether intestinal bacteria and bacteria-derived vesicles were directly administered to the intestines and passed through the intestinal mucosal barrier, it was confirmed that bacteria did not pass through the intestinal mucosal barrier, whereas bacteria-derived vesicles passed through the intestinal mucosal barrier (see Example 2).

In still another exemplary embodiment of the present invention, the pro-inflammatory effects of vesicles secreted from a Paracoccus zeaxanthinifaciens strain belonging to bacteria of the genus Paracoccus were evaluated by culturing the strain, and as a result of comparing the secretion levels of inflammatory mediators by treating macrophages with the Paracoccus zeaxanthinifaciens-derived vesicles at various concentrations, and then treating the macrophages with E. coli-derived vesicles, which are representative pathogenic vesicles, the ability of inflammatory mediators to be secreted was remarkably reduced by the Paracoccus zeaxanthinifaciens-derived vesicles as compared to the secretion of IL-6 and TNF-α by E. coli-derived vesicles (see Example 4).

In yet another exemplary embodiment of the present invention, the anti-inflammatory effects of vesicles derived from the Paracoccus zeaxanthinifaciens strain were evaluated, and as a result of evaluating the secretion of inflammatory mediators after treating macrophages with Paracoccus zeaxanthinifaciens-derived vesicles at various concentrations prior to treatment with E. coli-derived vesicles, which are pathogenic vesicles, it was confirmed that the Paracoccus zeaxanthinifaciens-derived vesicles efficiently inhibited the secretion of IL-6 and TNF-α by pro-inflammatory E. coli-derived vesicles (see Example 5).

Hereinafter, preferred Examples for helping the understanding of the present invention will be suggested. However, the following Examples are provided only to more easily understand the present invention, and the contents of the present invention are not limited by the following Examples.

### EXAMPLES

### Example 1. Analysis of In Vivo Absorption, Distribution, and Excretion Patterns of Gram-Negative Bacteria and Bacteria-Derived Vesicles

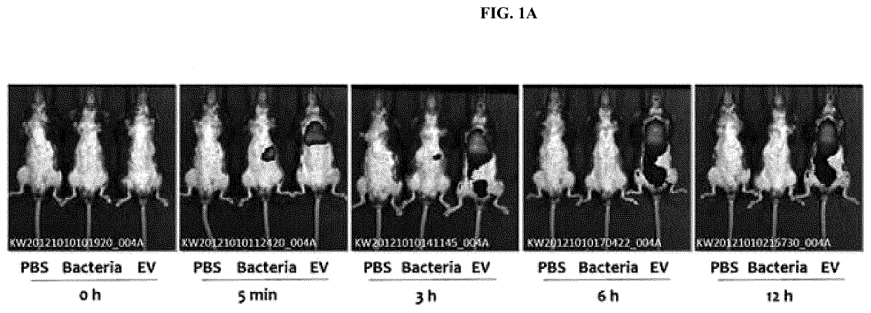

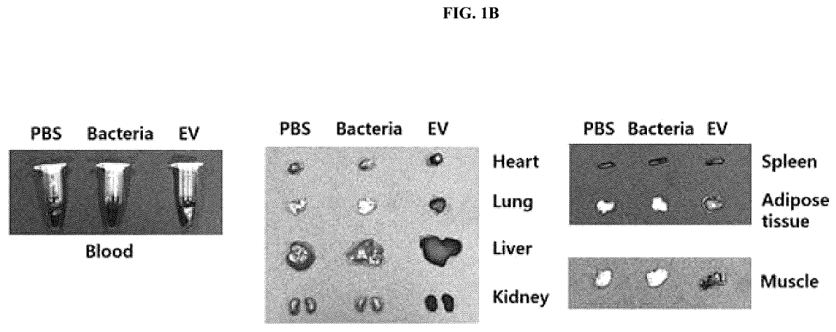

In order to evaluate whether Gram-negative bacteria such as bacteria of the genus Paracoccus and bacteria-derived vesicles are systemically absorbed through the gastrointestinal tract, an experiment was performed as follows. A dose of 50 μg of each of bacteria and bacteria-derived vesicles, both labeled with fluorescence, was administered to the gastrointestinal tract of a mouse, and fluorescence was measured 0 minute, 5 minutes, 3 hours, 6 hours, and 12 hours later. As a result of observing the entire image of the mouse, as shown in FIG. 1A, the bacteria were not systemically absorbed, but the bacteria-derived vesicles were systemically absorbed 5 minutes after administration, and strong fluorescence was observed in the bladder 3 minutes after administration, indicating that the vesicles were excreted through the urinary system, and in addition, it can be seen that the vesicles were also present in the body up to 12 hours after administration.

In order to evaluate the aspect in which the bacteria and the bacteria-derived vesicles infiltrate into various organs after they are systemically absorbed, 50 μg of bacteria and bacteria-derived vesicles, both labeled with fluorescence, were administered in the same manner as described above, and then the blood, heart, lungs, liver, kidneys, spleen, fat, and muscle were collected 12 hours after administration. As a result of observing fluorescence in the collected tissues, as shown in FIG. 1B, it can be seen that the vesicles derived from bacteria were distributed in the blood, heart, lungs, liver, kidneys, spleen, fat, muscle, and kidneys, but the bacteria were not absorbed.

### Example 2. Evaluation of Whether Gram-Negative Bacteria and Bacteria-Derived Vesicles Pass Through Intestinal Mucosal Barrier

In order to evaluate whether Gram-negative bacteria and bacteria-derived vesicles pass through the intestinal mucosal barrier and infiltrate into intestinal tissue, after bacteria and bacteria-derived vesicles were directly administered to the intestines, infiltration into intestinal tissue through the intestinal mucosal barrier was evaluated by an immunohistochemistry method. In order to evaluate the presence of bacteria and vesicles in the intestinal mucosa, antibodies against the bacteria and the vesicles were prepared, a green fluorescent protein (GFP) was attached and used, and after staining was performed with 4, 6-diamidino 2-phenylindole (DAPI), they were observed under a microscope.

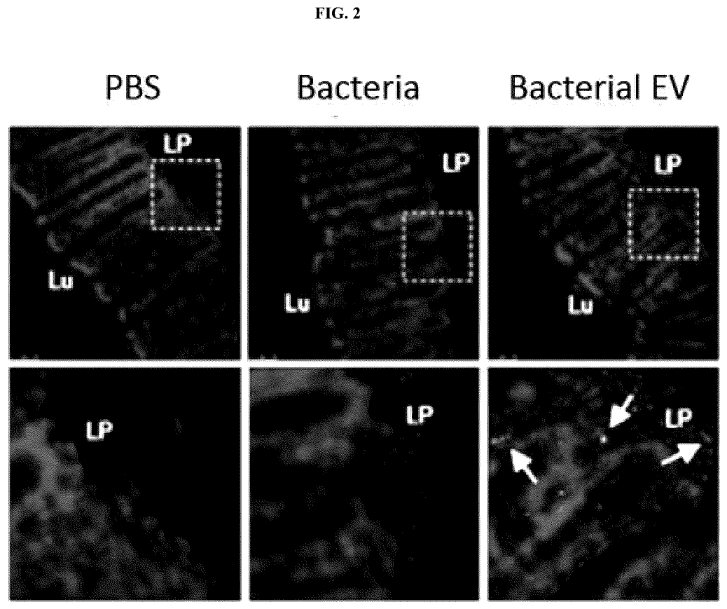

As a result, as shown in FIG. 2, it was confirmed that bacteria did not pass through the intestinal mucosal barrier, whereas bacteria-derived vesicles passed through the intestinal mucosa and infiltrated into intestinal tissue.

### Example 3. Isolation of Vesicles from Paracoccus zeaxanthinifaciens Culture Fluid

After culturing a Paracoccus zeaxanthinifaciens strain, vesicles thereof were isolated, analyzed and characterized. First, the Paracoccus zeaxanthinifaciens strain was cultured in a de Man, Rogosa and Sharpe (MRS) medium in an incubator at 37° C. until the absorbance (OD 600) became 1.0 to 1.5, and then sub-cultured in a Luria-Bertani (LB) medium. Thereafter, the culture solution including the strain was recovered and centrifuged at 10,000 g and 4ºC for 20 minutes to remove cells, and filtered with a 0.22-μm filter. The filtered supernatant was concentrated to a volume of 50 ml or less through microfiltration using a MasterFlex pump system (Cole-Parmer, US) with a 100 kDa Pellicon 2 Cassette filter membrane (Merck Millipore, US). The concentrated supernatant was filtered once again with a 0.22-μm filter. Thereafter, proteins were quantified using a bicinchoninic acid (BCA) assay, and the following experiments were performed on the obtained vesicles.

### Example 4. Pro-Inflammatory Effects of Paracoccus zeaxanthinifaciens-Derived Vesicles

To investigate the effects of Paracoccus zeaxanthinifaciens-derived vesicles (EV) on the secretion of inflammatory mediators (IL-6 and TNF-α) in inflammatory cells, Raw 264.7 cells, which are mouse macrophage cells, were treated with Paracoccus zeaxanthinifaciens-derived vesicles at various concentrations (0.1, 1, and 10 mg/ml), and then apoptosis measurement and ELISA were performed.

More specifically, Raw 264.7 cells aliquoted at 5×10⁴ cells/well into a 48-well cell culture plate were treated with Paracoccus zeaxanthinifaciens-derived vesicles at various concentrations, which were diluted with a Dulbecco's Minimum Essential Medium (DMEM) serum-free medium, and the treated cells were cultured for 12 hours. Thereafter, apoptosis was measured using EZ-CYTOX (Dogen, Korea), the cell culture solution was collected in a 1.5-ml tube and centrifuged at 3,000 g for 5 minutes, the supernatant was recovered and stored at −80° C., and then ELISA was performed.

To perform ELISA, a capture antibody was diluted with phosphate buffered saline (PBS), and 50 μl of the diluted solution was aliquoted into a 96-well polystyrene plate according to the working concentration, and then a reaction was carried out at 4° C. overnight. Thereafter, the plate was washed three times with 100 μl of a PBS containing 0.05% Tween-20 (PBST) solution, and blocking was performed at room temperature for 1 hour by aliquoting 100 μl of a PBS containing 1% BSA (RD) solution. 50 μl of each of the sample and the standard was aliquoted according to the concentration and allowed to react at room temperature for 2 hours.

Then, after washing three times with 100 μl of PBST, a detection antibody was diluted in RD, aliquoted at 50 μl according to the working concentration, and allowed to react at room temperature for 2 hours. Then, after washing three times with 100 μl of PBST, streptavidin-HRP (R&D Systems, USA) was diluted 1/40 in RD, aliquoted at 50 μl, and allowed to react at room temperature for 20 minutes.

Finally, after washing three times with 100 μl of PBST, 50 μl of a 3,3′,5,5′-tetramethylbenzidine (TMB) substrate (SurModics, USA) was aliquoted, and then when color development proceeded after 5 to 20 minutes, the reaction was stopped by aliquoting 50 μl of a 1 M sulfuric acid solution, and absorbance was measured at 450 nm using a SpectraMax M3 microplate reader (Molecular Devices, USA).

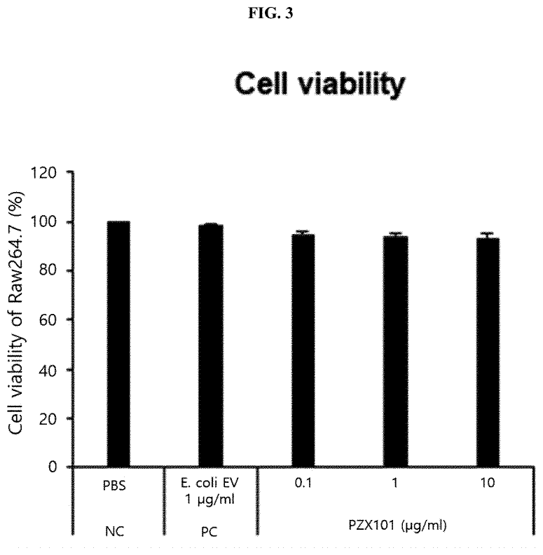

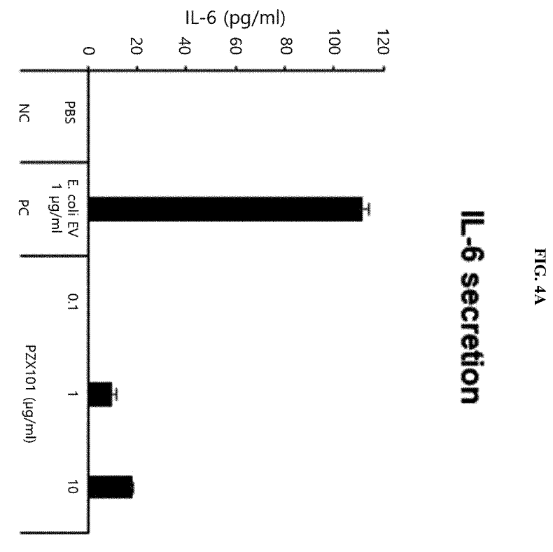

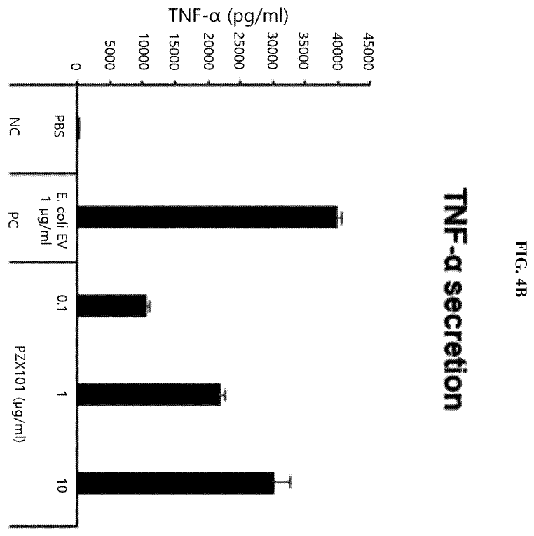

As a result, as shown in FIG. 3, apoptosis due to treatment with Paracoccus zeaxanthinifaciens-derived vesicles was not observed. Furthermore, as a result of evaluating the inflammatory mediator secretion pattern in inflammatory cells, as shown in FIGS. 4A and 4B, it was confirmed that the secretion of inflammatory mediators was significantly reduced upon treatment with Paracoccus zeaxanthinifaciens-derived vesicles compared to treatment with E. coli-derived vesicles (E. coli EV 1 μg/ml), which are a positive control.

### Example 5. Anti-Inflammatory Effects of Paracoccus zeaxanthinifaciens-Derived Vesicles

Based on the results of Example 4, in order to evaluate the anti-inflammatory effects of Paracoccus zeaxanthinifaciens-derived vesicles, after mouse macrophage cell lines were pre-treated with Paracoccus zeaxanthinifaciens-derived vesicles at various concentrations (0.1, 1, and 10 μg/ml) for 12 hours, the cell lines were treated with 1 μg/ml of E. coli-derived vesicles, which are a pathogenic factor, and then the secretion of inflammatory cytokines was measured by ELISA 12 hours later.

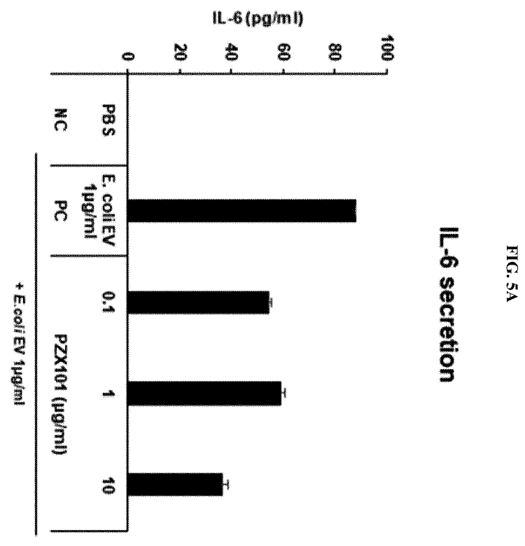

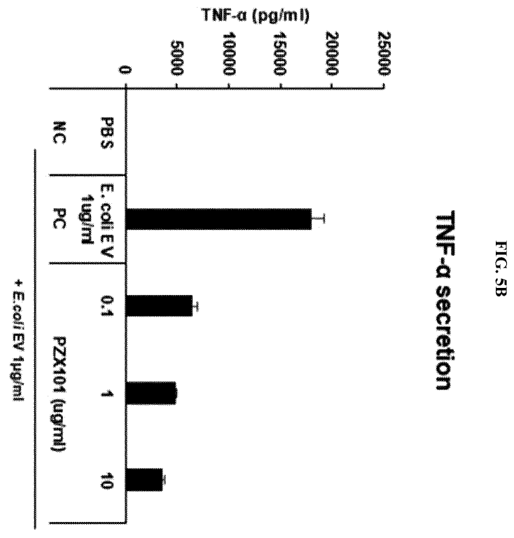

As a result, as shown in FIGS. 5A and 5B, it was confirmed that the amount of IL-6 and TNF-α secreted by inflammatory cells stimulated by E. coli-derived vesicles was remarkably suppressed upon treatment with Paracoccus zeaxanthinifaciens-derived vesicles. This means that Paracoccus zeaxanthinifaciens-derived vesicles can efficiently inhibit inflammatory responses induced by pathogenic factors such as E. coli-derived vesicles.

The above-described description of the present invention is provided for illustrative purposes, and those skilled in the art to which the present invention pertains will understand that the present invention can be easily modified into other specific forms without changing the technical spirit or essential features of the present invention. Therefore, it should be understood that the above-described examples are only exemplary in all aspects and are not restrictive.

### INDUSTRIAL APPLICABILITY

The vesicles derived from bacteria of the genus Paracoccus according to the present invention are expected to be advantageously used for the prevention, alleviation or treatment of inflammatory diseases, and thus have industrial applicability.

1.-21. (canceled)

22. A method for treating or alleviating an inflammatory disease, the method comprising administering a composition comprising vesicles derived from bacteria of the genus Paracoccus as an active ingredient to a subject in need thereof.

23. The method of claim 22, wherein the bacteria is Paracoccus zeaxanthinifaciens.

24. The method of claim 22, wherein the vesicles have an average diameter of 10 to 1000 nm.

25. The method of claim 22, wherein the vesicles are naturally secreted vesicles or artificially produced vesicles from bacteria of the genus Paracoccus.

26. The method of claim 22, wherein the inflammatory disease is selected from the group consisting of an inflammatory eye disease, an inflammatory oral disease, an inflammatory stomach disease, an inflammatory bowel disease, an inflammatory skin disease, an inflammatory respiratory disease, an inflammatory cardiovascular disease, an inflammatory brain disease, and combinations thereof.

27. The method of claim 26, wherein the inflammatory eye disease is selected from the group consisting of macular degeneration, diabetic retinopathy, glaucoma and combinations thereof.

28. The method of claim 26, wherein the inflammatory oral disease is selected from the group consisting of gingivitis, periodontitis, oral cancer, and combinations thereof.

29. The method of claim 26, wherein the inflammatory stomach disease is gastritis or gastric cancer.

30. The method of claim 26, wherein the inflammatory bowel disease is selected from the group consisting of colitis, food allergies, celiac disease, colon polyps, colorectal cancer, and combinations thereof.

31. The method of claim 26, wherein the inflammatory skin disease is atopic dermatitis or psoriasis.

32. The method of claim 26, wherein the inflammatory respiratory disease is selected from the group consisting of rhinitis, sinusitis, nasal polyps, asthma, chronic obstructive pulmonary disease, lung cancer, and combinations thereof.

33. The method of claim 26, wherein the inflammatory cardiovascular disease is selected from the group consisting of arteriosclerosis, angina pectoris, coronary artery disease, stroke, and combinations thereof.

34. The method of claim 26, wherein the inflammatory brain disease is selected from the group consisting of Alzheimer's disease, Parkinson's disease, Lou Gehrig's disease, depression, and combinations thereof.

35. The method of claim 22, wherein the composition is a pharmaceutical composition, a food composition, a cosmetic composition, an inhalant composition, or an ophthalmic composition.
